(19) United States
(12) Patent Application Publication
(10) Pub. No.: US 2026/0306789 A1
(43) Pub. Date: Oct. 8, 2026

(54) Method of Automatic Manufacturing a Tubular Device for a Catheter

(71) Applicant: BIOTRONIK SE & Co. KG, Berlin (DE)

(72) Inventor: Jan Helge RICHTER, Berlin (DE)

(73) Assignee: BIOTRONIK SE & Co. KG, Berlin (DE)

(21) Appl. No.: 19/482,542

(22) PCT Filed: May 17, 2024
(86) PCT No.: PCT/EP2024/063742

§ 371 Date: Nov. 7, 2025

(30) Foreign Application Priority Data
May 23, 2023 (EP) 23174740.3

(51) Int. Cl. A61M 25/00; A61M 25/01

(52) U.S. Cl. CPC A61M 25/0012; A61M 25/0045; A61M 25/0147; A61M 2207/10

(57) ABSTRACT

Method of manufacturing a tubular device for a catheter sized for introduction into a patient's body, including providing a mandrel having an outer sheath as well as a first end, a second end and a longitudinal axis; providing a solid wire consisting of elastic material; positioning an end section of the solid wire adjacent the first end of the mandrel such that it extends parallel to or around the longitudinal axis of the mandrel; and wrapping one or more reinforcement elements around the mandrel and around at least a pre-defined section of the solid wire with such tension that the one or more reinforcement elements are pressed into the surface of the solid wire. An assembly manufactured with the above method and a respective tubular device for a catheter are also disclosed.

### CROSS-REFERENCE TO RELATED APPLICATIONS

This application is the United States National Phase under 35 U.S.C. § 371 of PCT International Patent Application No. PCT/EP2024/063742, filed on May 17, 2024, which claims the benefit of European Patent Application No. 23174740.3, filed on May 23, 2023, the disclosures of which are hereby incorporated by reference herein in their entireties.

### BACKGROUND

Elongated tubular medical devices, such as diagnostic or treatment catheters or sheaths may be provided for introduction/insertion into a patient's body, e.g., into the patient's vasculature or other body cavities. For example, a catheter may have a distal portion configured to be introduced into a body cavity and advanced to one or more target locations within the patient's body by manipulating a proximal end of the catheter. Such catheters may be used, for example, for a cardiac pacemaker (e.g. implantable leadless pacemaker, ILP) or for a sensor for measuring a pulmonary artery pressure. Within the scope of the present invention, the term catheter or catheter tube shall be understood to refer to medical devices such as conventional catheters as well as sheaths, such as introducer sheaths for electrode leads for HIS bundle pacing.

Such catheter tubes usually comprise a tube wall, which surrounds one or more functional tube lumens. The tube wall can at least partially consist of a relatively flexible plastic material, such as polyether block amide (PEBA) or polyethylene (PE), so that the catheter tube can easily follow the course of the cavities of a patient. Depending on the application, a distal end of the catheter tube is, in general, to be guided to a particular target location in the body of the patient, for example so as to place an implant there, carry out diagnostics, or carry out a treatment, such as an ablation treatment or a targeted drug delivery. For these functions the catheter provides at least one functional inner lumen. A proximal end of the catheter tube can, for example, transition into a rigid catheter shaft, which remains outside the patient's body. For example, a health care practitioner (HCP) can grab a handle at the proximal end of the catheter shaft so as to advance the catheter or retract it.

In some known catheters (i.e. steerable catheters), in particular a distal portion of the catheter tube exhibits a certain steerability. This means, for example, that an orientation of the distal end of the catheter tube can be changed in a controlled manner, for example so as to position an implant in a targeted manner at a desired implantation site, or so as to act on the tissue of the patient in desired manner. The steering can be provided, for example, by the actuation of a steering mechanism at the handle at the proximal end of the catheter shaft. For example, document US 2022/0379079 A1 discloses a catheter tube for a steerable catheter comprising a wall with a mesh that forms a functional inner lumen and a guide lumen around which the mesh is braided. Within the guide lumen a pulling element extends from a proximal portion of the catheter tube to a distal portion of the catheter tube, wherein the pulling element is connected in a tension-resistant manner to the tube wall in the distal portion, and the guide lumen guiding the pulling element at least partially around the tube lumen. The distal portion of the known catheter tube is deformed by an actuation of the pulling element (e.g. a pulling wire).

During manufacturing of such steerable catheters often steps that allow discontinuous production only are used and several difficulties and high costs arise therefrom. Further, it is desired that catheter tube layers have a small wall thickness to provide greater functional and/or guide lumens. Further, it is observed that a supporting element (e.g. wire or mandrel) that is positioned within the catheter tube or its wall to keep the functional lumen and/or guide lumen open stick to the catheter wall material so that the supporting element cannot be removed from the catheter tube assembly. Thereby production costs are increased. Additionally, during use of the catheter tube, friction is generated between the guide lumen wall and the pulling element which needs to be reduced.

The present disclosure is directed toward overcoming one or more of the above-mentioned problems, though not necessarily limited to embodiments that do.

### SUMMARY

Accordingly, it is an object of the present invention to provide a manufacturing method and an assembly for a tubular device for a catheter that overcome above problems.

The above object is solved by a method of manufacturing a tubular device for a catheter with the features of claim 1, an assembly for a tubular device for a catheter having the features of claim 10 and a tubular device for a catheter having the features of claim 13.

In particular, the above object is solved by a method of manufacturing a tubular device for a catheter sized for insertion into a patient's body, comprising the following steps:
Providing a mandrel having an outer sheath as well as a first end, a second end and a longitudinal axis;
Providing a solid wire consisting of elastic material;
Positioning an end section of the solid wire adjacent the first end of the mandrel such that it extends parallel to and/or around the longitudinal axis of the mandrel;
Wrapping one or more reinforcement elements around the mandrel and around at least a pre-defined section of the solid wire with such tension that the one or more reinforcement elements are pressed into the surface of the solid wire and simultaneously supplying further sections of the solid wire parallel to the longitudinal axis of the mandrel until the second end of the mandrel or a pre-defined section of the mandrel is covered by the one or more reinforcement elements;
Cutting through the solid wire and the one or more reinforcement elements and, if applicable, the mandrel, thereby receiving an assembly;
Providing a cover layer and laminating this cover layer and/or providing a cover layer material and extruding this cover layer material onto the assembly; and
Removing the solid wire by pulling and removing the mandrel by pulling from the covered assembly.

The above manufacturing method provides an automatic assembly for a catheter for insertion into the patient's body which comprises an inner (central, functional) lumen for a medical device or sensor elements for carrying out a treatment or diagnostics created by the mandrel and comprises a guide lumen for a steering wire created by the solid wire. The inner lumen extends along a longitudinal axis (of the catheter tube or the mandrel). The guide lumen may be (at least partly) parallel to the longitudinal axis and/or may run (at least partly) around the longitudinal axis, e.g. helically around the longitudinal axis.

In one embodiment of the method, the guide lumen and/or the solid wire runs/extends—in particular helically—around the longitudinal axis at or along the distal portion of the catheter tube or of the mandrel, in particular only at or along the distal portion of the catheter tube or of the mandrel.

The one or more reinforcement elements form a layer that is created by wrapping (e.g. wounding or braiding), e.g. helically or pseudo-helically wrapping, around the inner lumen, i.e. forms at least partly the wall of the tubular device or the assembly therefor. The guide lumen is embedded within the wrapping (e.g. the braid, mesh, wrapping) of the tubular device or the assembly therefor. Due to the guide lumen for the steering wire being embedded into the wrapping of the tube wall, high dimensional stability (with good reproducibility of the shape) of the catheter tube can be achieved. In this way, high pushing or pulling forces can be transmitted from a proximal end into a distal end of the catheter tube. The catheter tube can thus additionally have a sufficiently rigid design, so as to allow, for example, secure guidance of a medical device.

In one embodiment, braiding may be provided, if three or more thread-like reinforcement elements are used, by a well-known braiding machine that interlaces the three or more reinforcement elements to create a tubular braid. Braids with right-and left-turning carriers of different numbers may be used, e.g. 8- or 16-fold wire braids in which 4 or 8 wires, respectively, are braided in opposite directions. A braided wire may consist of a flat wire or one or more round wires. For example, in the braiding machine three or more rotating car braider (or first horn gear braider) are located on a closed circle, wherein each horn gear braider is arranged such that the thread-like reinforcement within a braiding machine are passed from one horn gear track plate to the adjacent horn gear track plate. Thereby each reinforcement element is mounted on a bobbin at a spool carrier that is driven by one horn gear. In one embodiment, at least 3 (preferably 16, i.e. 2×8) reinforcement elements may be used.

In some embodiments, the braiding machine may have over 300 rotating clappers and many impellers, wherein each impeller may have a central feedthrough through which, as in a preferred embodiment, ropes (or other reinforcement elements) may be fed, which may then be braided longitudinally as so-called standing threads and serve as stiffening elements. The shape and number of the stiffening elements are arbitrary, the limiting factor is only the size of the feedthrough. Basically, it possible to design (the size of) each of these feedthroughs variably.

The mandrel supports the tubular device assembly during manufacturing and, after its removal, it leaves the inner (central, functional) lumen of the tubular device. The tubular or solid mandrel having the outer form of a wire or a rod comprises an outer sheath that stays within the tubular device assembly after removal of the mandrel. The used mandrel may have a dimension in longitudinal direction (i.e. the direction of its longitudinal axis) that is about the length of the assembly for one catheter from the beginning or may be longer and therefore cut to the pre-defined dimension (length) during manufacturing. The mandrel may consist of or may comprise a metal or plastic material, for example, at least one material of the group comprising stainless steel, polyether ether ketone (PEEK), polytetrafluoroethylene (PTFE), and any alloy of these materials. The mandrel may have a circular, an elliptical or an angular (triangular, square, pentagonal etc.) cross section outer profile.

In one embodiment of the method, the outer sheath of the mandrel is a polytetrafluoroethylene (PTFE) layer and/or the outer sheath comprises an anti-stick coating at its inner surface. This makes it easier to remove the mandrel from the assembly. The sheath remains as the inner layer of the assembly thereby forming the inner surface of the inner (functional) lumen. The outer sheath of the mandrel may comprise or consist of at least one material of the group comprising PTFE, PE, or other materials with a lubricious coating. For coating e.g. polyvinylpyrrolidone (PVP) may be used. PEBA or Polyamid (PA, e.g. PA 12) may serve as carrier film. In one embodiment, the outer surface of the outer sheath may be slightly etched, for example by an active Na-solvent, prior using the sheathed mandrel in the above method. Thereby the later bond with the cover layer during the lamination or extrusion step can be improved.

The one or more reinforcement elements are wrapped (e.g. wounded or braided) around the sheathed mandrel and around at least a pre-defined section of the solid wire. Accordingly, the solid wire may extend in and wrapped about a longitudinal dimension that corresponds to the dimension in longitudinal direction of the mandrel or in a pre-defined part of this dimension. Accordingly, the solid wire may extend outside the braid formed by the one or more reinforcement elements.

In one embodiment of the method, the solid wire may extend outside the braid, i.e. outside the reinforcement elements, at or along a portion of the catheter tube, in particular at or along the proximal portion and/or along an intermediate portion between the proximal and distal portion of the catheter tube or the mandrel. Catheters have a long, largely straight and/or comparatively rigid area in the proximal and/or intermediate portion. In contrast to the distal portion (e.g. distal deflection portion), such a proximal and/or intermediate portion may dispense with a special supporting effect of the reinforcement elements and/or friction-minimizing measures, which simplifies the manufacturing process. In addition, the solid wire may be removed more easily if it is not wrapped by the braid and therefore not clamped.

One reinforcement element may be a rope, a wire or a filament having a circular, an elliptical or an angular (triangular, square, pentagonal etc.) cross section profile. One reinforcement element may be a single filament or a multi filament wire. One reinforcement element may have a coating or an outer sheath, for example, consisting of PTFE, PEBA, PE. Preferably, the wire mesh/braid comprises stainless steel round wires, which are triple-guided (3 wires on each spool, lying next to each other). The material of such reinforcement element (filament) may comprise or consist of at least one material of the group comprising an aromatic polyamide (e.g. Kevlar) and other polyamid types, polyether ether ketone (PEEK), NiCoCrMo-alloy (e.g. MP35N), stainless steel, polyurethane (PU), and polyether block amide (PEBA, e.g. PEBAX, such as Pebax 7333, Pebax 6333, Pebax 5533, Pebax 4533 or Pebax 3533). The one or more reinforcement elements reinforce the tubular device and increase torsional stiffness and buckling resistance. If at least two reinforcement elements are used, reinforcement elements of different materials may be combined. Further, if one reinforcement element is a multi filament wire, the at least two filaments may consist of different materials.

In one embodiment, the tubular device may comprise different portions that differ in their bending stiffness or rigidity. The bending stiffness may be adjusted by the number of intersecting points of the braid or the pitch of the helix or helices (e.g. a double helix) of the one or more reinforcement elements wound around the sheathed mandrel. The number of intersecting points and pitch may be adjusted by the velocity with which the sheathed mandrel is moved relative to the horn gears of the braiding machine during braiding. In particular, the bending flexibility is greater if the number of intersecting points per millimeter of intersecting reinforcement elements in longitudinal direction is greater or the pitch is smaller.

The tubular device further comprises a solid wire consisting of elastic material that is wrapped at least around its pre-defined section and later removed by pulling. Further, the one or more reinforcement elements are wrapped around the solid wire with such tension that the one or more reinforcement elements are pressed into the surface of the solid wire that after removal of the solid wire the reinforcement elements form transversally running projections at the inner surface of the residual lumen (i.e. the guide lumen), e.g. helical or pseudo-helical projections. The solid wire is fully solid, elastic and comprises or consists of at least one material of the group comprising PTFE, PEEK, flourinated ethylene propylene (FEP), silicone.

If the one or more reinforcement elements are fully wrapped around the mandrel or along a pre-defined sufficient length of the mandrel, the wrapping (i.e. the reinforcement elements) and the solid wire, and, if applicable, the at least one support wire is cut by a cutting tool. The cutting tool may be activated and moved relative to the assembly to the pre-defined position, where the cutting will be provided.

After cutting, the assembly is moved to the lamination unit or extruding unit. For that, the assembly is grabbed and moved, wherein the cut reinforcement element(s) on the end which is wrapped at last is/are temporarily fixed by an adhesive tape. Then, in one embodiment, the assembly is moved into/inserted into at least one tube or tubular element comprising the material of the cover layer. In one embodiment, the inner surface of this tube/tubular element may comprise an activation or an adhesive material. The tube/tubular element may have a single layer or at least two layers. The cover layer tube/tubular element is then laminated onto the assembly such that it forms the outermost layer of this assembly. Alternatively, the cover layer material is extruded onto the assembly.

In one embodiment, the assembly is inserted into several cover layer tubes or tube elements such that these tubes/elements are arranged adjacently in longitudinal direction, wherein at least two of these tubes have different materials or material properties. The cover layer may consist of or comprise at least one material of the group comprising PEBA, PU, PA, PE, PET, silicone, copolymers comprising silicone, PU and/or polyimide (PI). For example, the following material and/or property variations with regard to the cover layer of the assembly may be used: materials composition, shore hardness, friction property. A glass transition or melting temperature may be another selection criterion, e.g. if the catheter should be softer at body temperature. With regard to the position, where the solid wire penetrates and projects from the wrapping, one section of the cover layer tube is located on one side and the second section of the cover layer tube is located on the other side (of this position) thereby leaving a short (if measured in longitudinal direction) recess in longitudinal direction. During lamination, the cover layer material flows into this recess thereby filling this recess. The lamination temperature may be, for example, between 140° C. and 270° C.

The lamination step comprises providing higher temperature and pressure such that the cover material is fixedly connected to the reinforcement elements and the sheath material of the mandrel sheath, in particular by a substance-to-substance bond and, if applicable, also by a positive-locking connection. The molten or softened cover material flows into the free space between the reinforcement elements and fills this space as well as the recess caused by the position where the solid wire is lead out through the wrapping and between two neighboring cover layer tubes or tube elements. However, the solid wire provides the guide lumen through wrapping and the cover layer, as well, for the steering wire.

The alternative or additional extrusion process comprises extrusion of the material onto the outer surface of the assembly. The usable cover layer material corresponds to the material mentioned above with regard to the lamination step. The extrusion step may be applied instead of the lamination step or additionally, namely to the outer surface of the laminated cover layer. The temperature to which the cover layer material is heated during extrusion may be, for example, between 130° C. and 250° C., preferably between 165° C. and 195° C., and the pressure at which the material is pressed through a pre-defined die having, for example, a circular cross section may depend on the temperature at the die, and on the extrusion speed. In the case of extrusion, the outer contour (of the cover layer) depends on the shape of the extrusion die, this may have any shape. If the lamination process is chosen by means of shrink tubing, a circular/round shape is created. PEBA types (3533-7033) and PA can be laminated in an oven at a temperature of 193° C. Shrinking of a FEP heat shrinkable tubing (shrink tubing) may be done at a temperature of at least 170° C. The extrusion step may be repeated using the same or different materials thereby forming a composed cover layer. As a result, the outer surface of the sheathed mandrel with the one or more reinforcement elements is covered by the laminated and/or extruded cover layer.

After application of the cover layer, the mandrel is pulled and thereby removed from the assembly. Similarly, the solid wire, is pulled and thereby removed from the assembly. During pulling the mandrel may be inserted into a perforated plate so that the mandrel can be pulled out. The assembly may then be stacked in a pre-defined box and stored for further process steps for tubular device manufacturing. The force to pull out the mandrel may be between 10 N and 40 N. A clamping device may be used for the mandrel and at the same time a stripper may be pressed against one end face of the shaft via the mandrel.

The above method reduces production costs as the mandrel and the solid wire (the solid wire consisting of elastic material) can easily be removed from the assembly. The elastic material does not stick to the material of the reinforcement elements and the material of the cover layer. Additionally, the elasticity of the solid wire facilitates removal since the diameter of the solid wire is reduced when it is pulled during removal. Hence, production costs are reduced. Additional, continuous manufacturing is possible with the above steps so that the production costs are further reduced as described in more detail below. Furthermore, the one or more reinforcement elements that are pressed into the surface of the solid wire form projections (e.g. transversal, for example helically or pseudo-helically, projections) at the inner wall of the guide lumen when the solid wire is removed. Accordingly, the friction between a steering wire that is movably located within the guide lumen when using the assembly for a catheter and the inner wall of the guide lumen is reduced as the steering wire slides along the helical projections only and does not come into contact with at least a part of the remaining wand portion as it is recessed.

In one embodiment, the solid wire may have a pre-defined circular, an elliptical or an angular (triangular, square, pentagonal etc.) cross section profile. The cross section profile determines the form of the guide lumen.

In one embodiment of the method, the solid wire is an elastomer having a Young's modulus below 0,6 GPa or below 500 MPa, for example, below 430 MPa, and a glass transition or melting temperature that is higher than the one of the cover layer material or the temperature used during the lamination and/or extrusion step. Such elastic material is particularly suitable for removal after the assembly is formed. The high glass transition or melting temperature of the solid wire material (compared to the one of the cover layer) ensures that the solid wire is not fixedly connected to the cover layer during the lamination step and therefore can easily be removed after this step. For example, the glass transition temperature or melting temperature of the solid wire material is between 300° C. and 350° C., in particular 327° C..

In one embodiment of the method, as described above, at least three reinforcement elements are used and provided by one or more rotating first horn gears (also called first carriers) of a braiding machine which may be configured such that they rotate about their own central axis and about the longitudinal axis of the mandrel.

In one embodiment of the method, the solid wire is provided by a second horn gear (also called second carrier) of a braiding machine, wherein the second carrier is configured such that it is movable in radial direction with regard to the longitudinal axis of the mandrel. Accordingly, if the second carrier is in a pre-defined first radial position-the braiding machine wraps the solid wire and the mandrel having an outer sheath simultaneously thereby forming the assembly. In case the second carrier of the machine has a pre-defined first radial distance from the first radial position and away from the longitudinal axis, the solid wire is not wrapped by the one or more reinforcement elements so that it is located at the outer surface of the braid.

In one embodiment of the method, at least one support wire is provided simultaneously with the solid wire and parallel to and/or around the longitudinal axis of the mandrel (e.g. helically). The support wire is located adjacent the outer surface of the sheathed mandrel and is wrapped by the one or more reinforcement elements simultaneously with the sheath of the mandrel.

In one embodiment of the method, the possibly helical and/or deflectable distal portion is constructed separately from the stiffer proximal portion. The different portions (e.g. distal and proximal portion) may be produced separately and endlessly, then cut to any desired length and then joined together (e.g. by a welding process). A stretchable, endless and/or over-extruded core wire (e.g. the mandrel rod) from the roll is used (instead of straight, individual braiding cores onto which a PTFE inner liner tube is pushed), which can be rewound behind the braiding head and over-extruded (in a separate process or directly). Subsequently, (distal) segments of any length may be cut and the stretchable core wire and the solid wire, which may also be stretched, may be removed.

In a further embodiment, an extruded, rigid two-lumen tube (with the same geometry of the cross-sectional area as that of the distal portion, and e.g. made of polyamide 12) may be used. By using welding mandrels, the lumens may be kept open when welding the distal to the proximal portion. Ultrasonic, hot air and/or infrared welding may be used as a welding process e.g. in conjunction with a lost outer mold (e.g. FEP shrink tubing). High-frequency welding in a fixed outer mold is also possible, for example, to join distally and proximally different outer geometries with the same inner geometry. There may be, for example, a proximal and/or intermediate portion with a larger outer diameter than the distal portion with a correspondingly smaller outer diameter. The portion with a smaller outer geometry may used the ‘free space’ for further electrodes (e.g. ring electrodes). Different internal geometries may also be realized with appropriately designed welding cores.

In a further embodiment, an extruded, rigid single-lumen tube (with a different geometry of the cross-sectional area than that of the distal portion, and e.g. made of polyamide 12) or hyper tube may be used. In this case, the pull wire is guided in the proximal portion in the main lumen (e.g. the inner lumen) and thus closer to the imaginary axis. This is possible if irrigation of the catheter is not necessary or carried out using e.g. a second inner catheter or an additional irrigation tube. The advantage here is that the curvature tendency of the proximal portion is reduced when deflecting in the direction of deflection and thus the required stroke length of the steering wire for deflection of the distal area is shortened. Thus, shorter handle designs are possible.

In a further embodiment, a single-lumen tube and a two-lumen tube (e.g. each as described above) are connected so that the two lumens of the two-lumen tube open or merge into the one lumen of the single-lumen tube.

The above object is further solved, in particular, by an assembly for a tubular device for a catheter sized for insertion into a patient's body, comprising several layers, namely
a hollow cylindrical inner liner forming an inner (functional) lumen and having a longitudinal axis,
a braid or wrapped component of one or more reinforcement elements wrapped around the inner liner,
a cover layer surrounding the braid or wrapped component, and
a guide lumen,
   wherein the guide lumen is provided parallel to and/or around the longitudinal axis, wherein the guide lumen is arranged between the inner liner and the cover layer and is wrapped by at least a pre-defined part of the one or more reinforcement elements, wherein the one or more reinforcement elements form, e.g. helical, projections at the inner surface of the guide lumen. The above assembly may realize the above described embodiments and advantages, wherein the inner liner is the sheath of the mandrel and the cover layer may be manufactured by lamination or extrusion, for example. The inner lumen is created by the mandrel when it is removed and the guide lumen is created by the solid wire when it is removed.

In one embodiment of the assembly, the guide lumen extends in longitudinal direction along a pre-defined section of the inner lumen.

In one embodiment of the assembly, the assembly comprises at least one support wire extending parallel to and/or around the longitudinal axis (of the mandrel and/or the catheter tube) and is wrapped by at least a pre-defined part of the one or more reinforcement elements.

In one embodiment of the assembly, the at least one support wire extending—in particular helically—around the longitudinal axis at or along the distal portion of the catheter tube, in particular only at or along the distal portion of the catheter tube or of the mandrel.

The above object is further solved, in particular, by a tubular device for a steerable catheter sized for introduction into a patient's body comprising the above described assembly, wherein a steering wire is located within the guide lumen. Similarly, the tubular device comprises the assembly embodiments explained above and has the respective advantages.

In one embodiment of the device, a distal end of the steering wire that extends through the guide lumen from a distal end to a proximal end and is fixed to the distal end of the catheter, wherein the catheter comprises an actuator at its proximal end, wherein the actuator is coupled to a proximal end of the steering wire such that actuation (i.e. pushing or pulling) of the steering wire causes bending or change in direction of the distal end of the catheter. This allows steering of the catheter or sheath in an easy way.

In one embodiment of the tube device, the guide lumen runs/extends—in particular helically—around the longitudinal axis at or along the distal portion of the tube device, in particular only at or along the distal portion of the device, e.g. a catheter tube.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawings, wherein

FIG. 1 shows an embodiment of a catheter in a side view,

FIGS. 2 and 4 depict a first embodiment of an assembly during manufacturing in a first cross section,

FIG. 3 shows the embodiment of the assembly of FIG. 2 during manufacturing in a second cross section,

FIG. 5 shows a second embodiment of an assembly during manufacturing in a cross section,

FIG. 6 depicts a third embodiment of an assembly during a first manufacturing step in a cross section,

FIG. 6A shows a portion of the embodiment of the assembly of FIG. 6 during the same manufacturing step in a side view,

FIG. 7 depicts the embodiment of the assembly of FIG. 6 during a second manufacturing step in a cross section,

FIG. 8 to 10 show three different possibilities of wrapping two reinforcement elements for the embodiment of the assembly of FIG. 6 in a side view,

FIG. 11 to 12 depicts the embodiment of the assembly of FIG. 6 during a third manufacturing step in a cross section (FIG. 11) and in a longitudinal section along A-A in FIG. 11 (FIG. 12),

FIG. 13 shows the embodiment of the assembly of FIG. 6 during a fourth manufacturing step in a side view,

FIG. 14 depicts the embodiment of the assembly of FIG. 2 during manufacturing in a third cross section,

FIG. 15 shows an embodiment of a manufacturing setup for manufacturing of an embodiment of the assembly of FIG. 6 in a side view, and

FIG. 16 depicts an element of the manufacturing setup of FIG. 15 in a front view.

### DETAILED DESCRIPTION

Referring to the drawings, FIG. 1 shows an embodiment of a catheter having an elongated tubular device 1 with a longitudinal axis 2 and a proximal end la as well as a catheter tip 3 at its distal end. The catheter tip 3 may contain an element for patient's treatment and/or diagnosis, for example an ILP prepared to release. At the proximal end la of the tubular device 1 a handle 5 is provided which can be used by an HCP for introduction of the catheter into the patient's body, e.g. the patient's vasculature. Additionally, the tubular device 1 comprises at its proximal end la an actuator 7 that is connected with a proximal end of a steering wire 9 which proximal section extends outside the tubular device 1. The central and distal section of the steering wire 9 is located within a guide lumen of the tubular device 1 and is fixedly connected to a pre-defined location along the tubular device 1, in this case to the catheter tip 3. By actuation of the actuator 7, the steering wire (pull wire) 9 may be pulled and/or pushed thereby manipulating the catheter tip 3 in a pre-defined manner, e.g. bending or changing direction of the catheter tip.

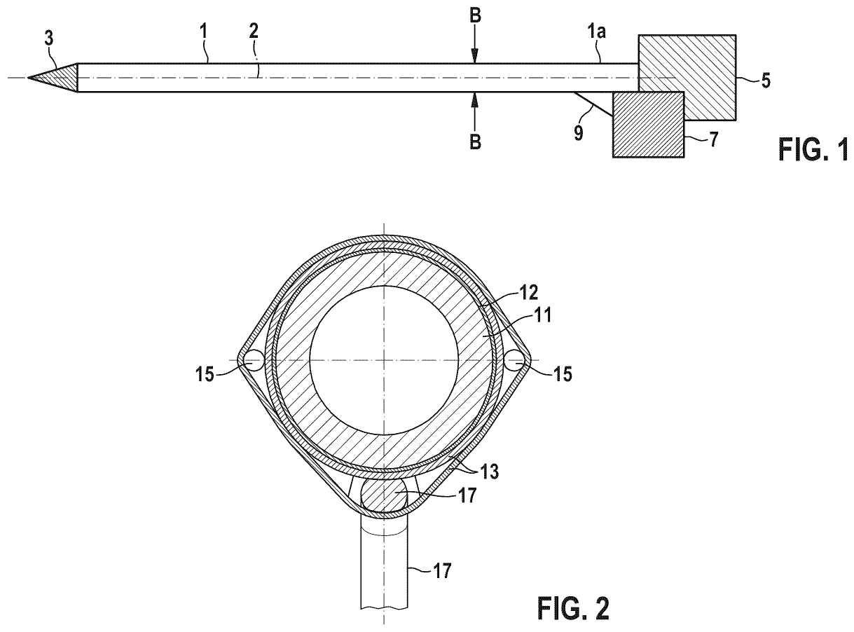

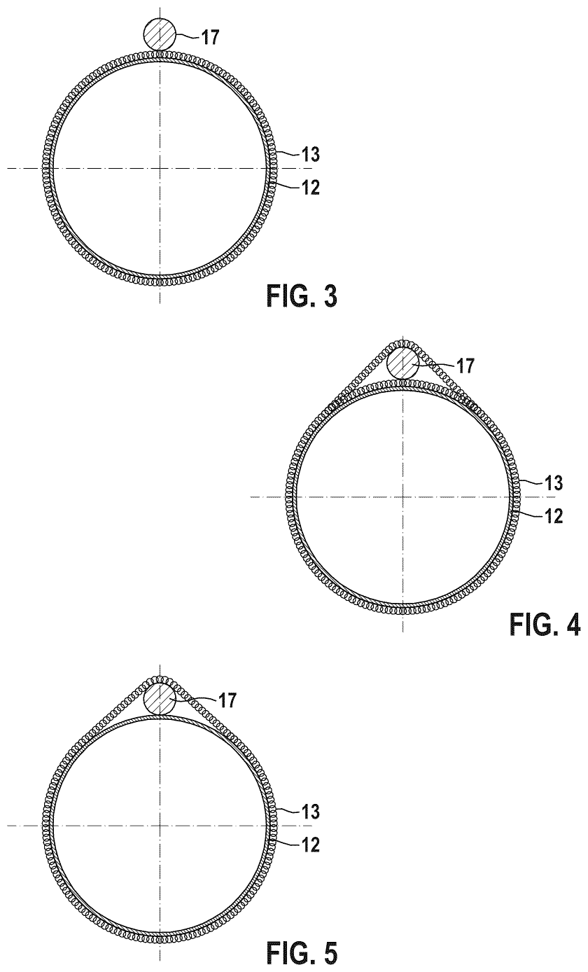

As indicated above, FIG. 2 to 4 and 14 show a first embodiment of an assembly for a tubular device of such catheter during a manufacturing step. In this step, the assembly comprises a tubular mandrel 11 consisting of stainless steel, wherein the mandrel 11 has an outer PTFE sheath 12. The thickness of the PTFE sheath 12 is, for example, 0,5 mm. After providing such sheathed mandrel 11, a braid/mesh 13 is wrapped around the sheath 12 such that two support wires 15 extending parallel to the longitudinal axis of the mandrel 11 and a solid wire 17 is wrapped by the braid 13, as well. The braid 13 extends over and under the solid wire 17 and each support wire 15 as shown in FIGS. 2 and 4. In another section of this tubular device, the solid wire 17 extends outside the braid 13, for example in a proximal section and/or at the proximal end of the assembly, as shown in FIG. 3. FIG. 5 depicts the possibility with regard to a second embodiment of an assembly for a tubular device where the braid 13 wraps over the solid wire 17 and not, as shown in FIG. 5, between the solid wire 17 and the sheath 12 of the mandrel 11 (i.e. does not wrap under the solid wire 17). The support wire may consist of stainless steel.

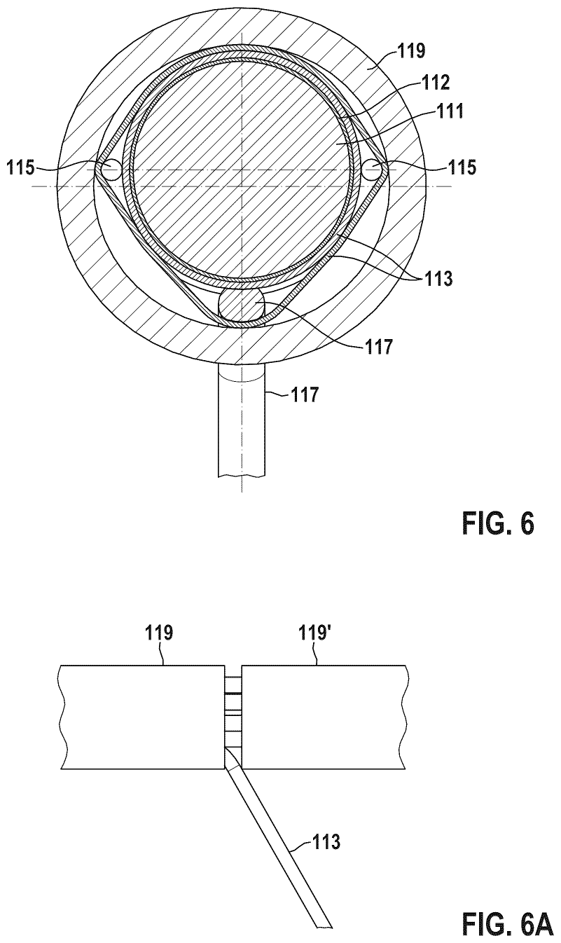

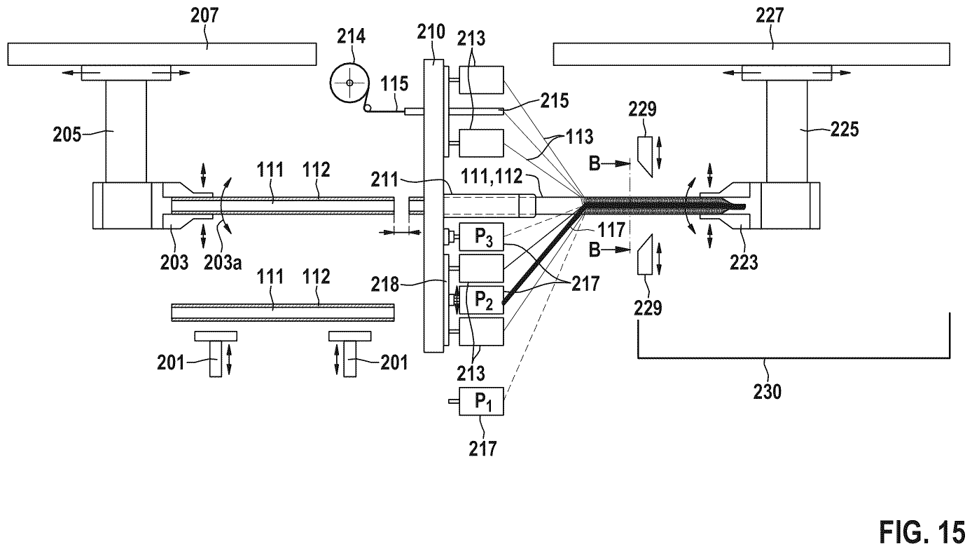

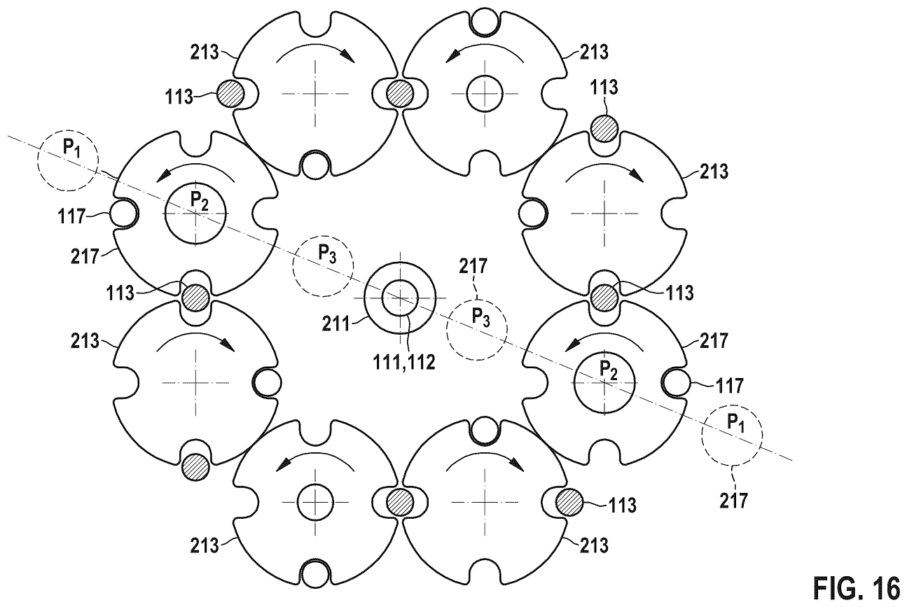

The whole manufacturing method of an assembly for a tubular device is now described with reference to a third embodiment of an assembly for a tubular device shown in FIGS. 6, 6A, and 7 to 16. A setup for manufacturing is shown in FIGS. 15 and 16.

The manufacturing method starts on the lower left-hand side of the setup shown in FIG. 15. A solid mandrel rod 111 consisting of stainless steel having a PTFE sheath 112 with a thickness between 0,3 and 0,8 mm, in particular of 0,5 mm is provided by a holding unit 201 that may be moved in vertical direction (see vertical arrows beside the holding unit 201) for handing over a single mandrel with a pre-defined length (i.e. dimension in longitudinal direction that extends along the longitudinal axis of the mandrel) to a grab 203. The grab 203 comprises elements that can be moved towards each other in vertical direction (see vertical arrows) such that the mandrel 111 is securely fixed within the grab 203. The grab 203 is further configured to rotate the mandrel 111 about its longitudinal axis (see arrow 203a). The grab 203 and thereby the sheathed mandrel 111 is movable by a holder 205 along a horizontal rail 207. In FIG. 15, the mandrel 111 is drawn with a distance from the holding unit 201 for clarity reasons, wherein the mandrel 111 is located on the holding unit 201 prior use in the manufacturing process.

For automatic, continuous manufacturing of tubular devices one sheathed mandrel 111 is stored on the holding unit 201, a second mandrel 111 is held by the grab 203 and a third mandrel 111 is processed within the braiding unit as shown on the right-hand side. After wrapping the braid, the assemblies are moved to and stored in a respective container 230 located on the lower right-hand side of the manufacturing setup.

For braiding the sheathed mandrel 111 is inserted into the braiding unit through a central opening 211 (see FIG. 16) within a holding disc 210. The holding disc 210 comprises six first horn gears 213, wherein each first horn gear 213 carries one reinforcement element 113 bobbin. Further, the holding disc 210 comprises two second horn gears 217 wherein each second horn gear 217 carries one solid wire 117 bobbin. To understand, one of the second horn gears 217 carries the carrier with the wire from the wire mesh (reinforcing element 113) and the other of the second horn gears 217 is necessary to feed the reinforcing elements or the solid material/wire 117. The second horn gears 217 may be alternately used, i.e. only one second horn gear 217 is used at one time. Further, the holding disc 210 comprises two openings, each with an elongated tubular element 215 through which a support wire 115 is provided from a spool 214 located in the back of the holding disc 210. This tubular element 215 is not shown in FIG. 16 due to clarity reasons.

As shown in FIG. 16, the first horn gears 213 and the second horn gears 217 rotate about their own axis, wherein, as generally known from horn gear braiding machines, their notched plate lead the respective bobbins along a quasi-sinusoidal path so that a mesh or braid is created from the reinforcement elements 113. In the configuration shown in FIG. 16 where both second horn gears 217 are arranged on the circular path of the first horn gears 213 (see positions P2 in FIG. 16), the solid wire 117 is wrapped by the reinforcement elements 113 such that it is located above the solid wire 117 and between the solid wire 117 and the sheath 112 of the mandrel 110 as depicted in FIGS. 2, 4, 6, 7. The solid wire 117 is included in the braid when the respective second horn gear 217 has this configuration. Additionally, the reinforcement elements 113 are braided with such tension (or tensioning force) that the reinforcement elements 113 are pressed into the surface of the solid wire 117 so that they slightly deform the surface of the solid wire 117 by this pressure. The tensioning force of the braided wires may be realized by springs, which preferably have tensioning forces between 7 N and 20 N. The tensioning force of the braided wires is determined by the tensioning force of the springs. With thicker wires, however, the tensioning force can also be significantly higher. Each reinforcement element 113 causes an indentation in the surface of the solid wire, i.e. the respective reinforcement element 113 (at least slightly) grooves the surface at its position due to the elasticity of the solid wire.

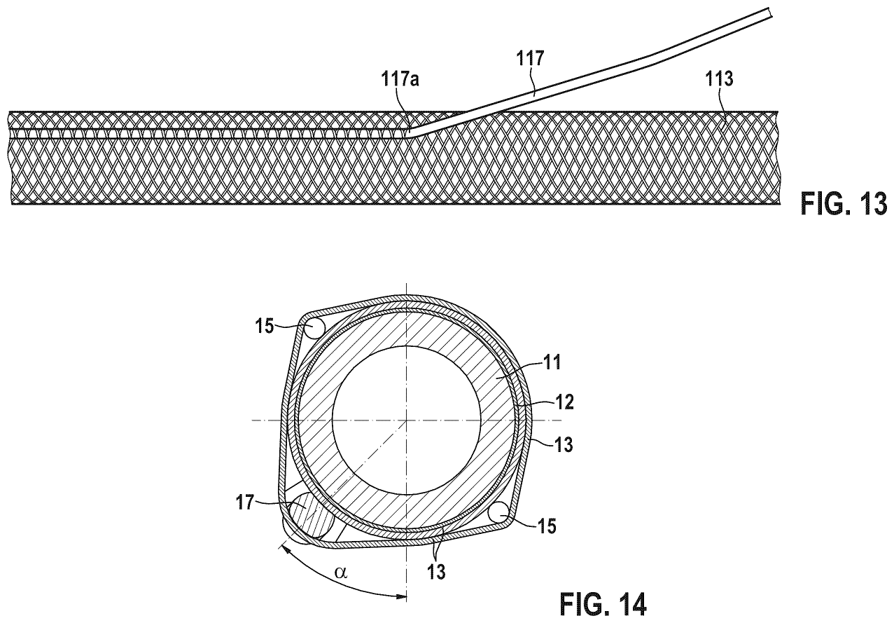

If the second horn gear 217 is moved in radial direction such that the second horn gear 217 is located within the circular line that may be drawn through the rotation centers of the first horn gears 213 (i.e. shown in FIG. 16 at position P3), the mesh/braid is located above the solid wire 117, only as shown for the solid wire 17 in FIG. 5. There is another position (i.e. position P1 in FIG. 16) in which the solid wire 117 is not covered by the mesh/braid produced by the six first horn gears 213. The solid wire runs/extends outside the braid as shown with regard to the solid wire 17 in FIG. 3. Thus, the solid wire 117 may run outside the braid at or along a portion of the catheter tube, in particular at or along the proximal portion and/or along an intermediate portion between the proximal and distal portion of the catheter tube. It is possible to move each second horn gear 217 in radial direction such that each one can take the first position P1, the second position P2 and the third position P3. When it is desired to change the location of the solid wire 117 with regard to the braid/mesh 113, the braiding machine is stopped and the position of the second horn gear 217 is changed to the desired location. After that, the braiding machine may be reactivated and the halted first horn gear 213 may work as indicated above. An assembly with a solid wire 117 wrapped with a braid 113 is shown in FIG. 13. The mesh was braided starting at the left-hand side using the second horn gear at the second position P2. When braiding arrived approximately in the center of this Fig., the position of the second horn gear 217 was changed from the position P2 to the position P1 (see FIG. 16). Accordingly, the solid wire 117 passes through the braid 113 at a location referred to as 117a and accordingly projects from the braid 113 as shown in FIG. 13 along the rest of the assembly (right-hand side from the point 117a). The solid wire 117 may be a PTFE wire having a thickness between 0,3 mm and 1,0 mm, in particular of 0,6 mm and a Young's modulus between 550 MPa and 600 MPa, in particular of 575 MPa. As reinforcement elements 113 a three-core metal wire of stainless steel (that may comprise an outer coating) may be used. The support wire 115 may consist of stainless steel.

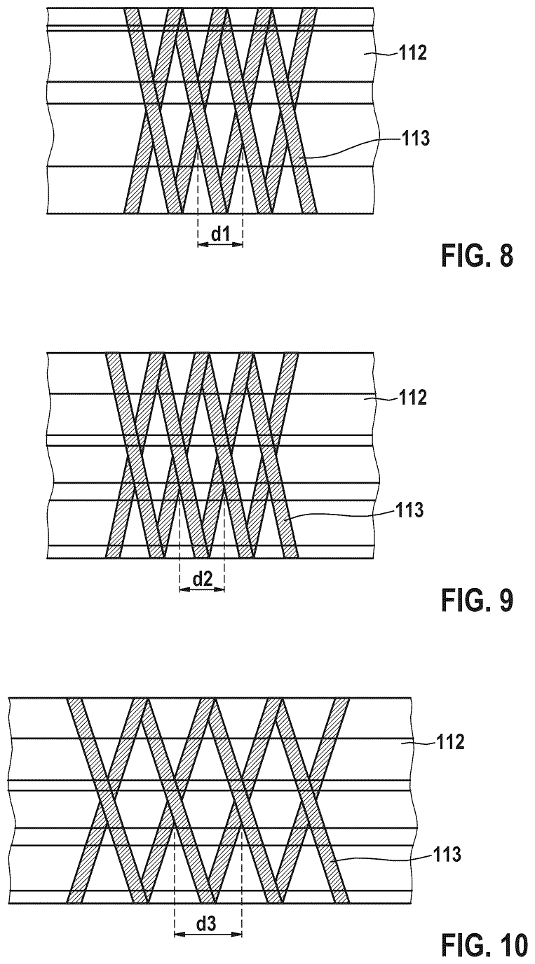

Further, the braiding machine starts braiding at the first end of the sheathed mandrel 111 and may form braids having a different number of intersecting points within one pre-defined length unit (into the direction of the longitudinal axis of the mandrel 111, e.g. 1 mm) or the distance of two neighboring intersecting points of the reinforcement elements is different as shown in FIG. 8 to 10. In these Figures, for clarity reasons only two reinforcement elements are drawn as a braid, wherein in FIG. 8 the distance of two neighboring intersecting points of the two reinforcement elements is d1, in FIG. 9 the distance is d2 and in FIG. 10 the distance is d3, wherein d1<d2<d3. The bending flexibility is greatest if the distance of neighboring intersecting points is lowest, i.e. in the configuration shown in FIG. 8. For example, a distal braid section may comprise a low distance of two neighboring intersecting points and thereby a great bending flexibility for steering, whereas at the distal end the braid may be provided such that the distance of two neighboring intersecting points is greater.

During braiding, a second grab 223 located opposite the braiding unit comprising the holding disc 210 receives the first end of the braided mandrel 111. The second grab 223 may be moved by its holder 225 along a rail 227 in horizontal direction.

After wrapping the braid 113 around the sheathed mandrel 111, the solid wire 117 and two support wires 115, wherein the support wires 115 are located with a circumferential distance to the solid wire 117 of 90°, the braiding is stopped when the second end of the sheathing mandrel 111 is fully covered by the braid. Then, the second grab 223 moves the mandrel 111 in horizontal direction away from the braiding unit and the braid 113 is then cut by vertically movable cutting tools 229. Then, the braid covered mandrel 111 is moved to the container 230.

From the container 230 the covered mandrel 111 is moved to lamination unit (not shown). There, the assembly having the sheathed mandrel 111, two support wires 115, one solid wire 117 and the braid 113 is inserted into at least one cover layer tube 119, 119′ consisting of PEBA and having a wall thickness between 0,2 and 0,8 mm, in particular of 0,5 mm. This assembly after insertion into the cover layer tube 119 is shown in FIG. 6. As one can derive from FIG. 6a, different cover layer tube sections 119, 119′ may be used along the whole length of the assembly (i.e. the length is the dimension in the direction of the longitudinal axis) in order to realize different properties of the assembly along the whole length.

In the next step, the cover layer tube 119 is laminated onto the underlying layers, i.e. the sheath 112 of the mandrel 111 and the braid 113. The lamination is conducted at a temperature between 140° C. and 300° C. The pressure during lamination is not precisely defined. FEP shrink tubes are used, and the pressure exerted by these shrink tubes depends on various factors, e.g. shrink ratio, wall thickness, degree of crosslinking and shrink temperature. For example, when shrinking in the laminator, the applied temperature is higher than the melting temperature of cover layer tubes (PEBA tubes), because a high temperature allows a high travel speed in the laminator. The shrink tube shrinks optimally between 170° C. and 215° C., and the absorbed amount of heat is not transferred to the cover layer tubes (PEBA tubes) until the shrink tube is unshrunk. The melting temperatures of the jacket tubes are then between 142° C. and 178° C. A temperature of 300° C. should not be exceeded, otherwise toxic vapors may be released by the FEP shrink tubes.

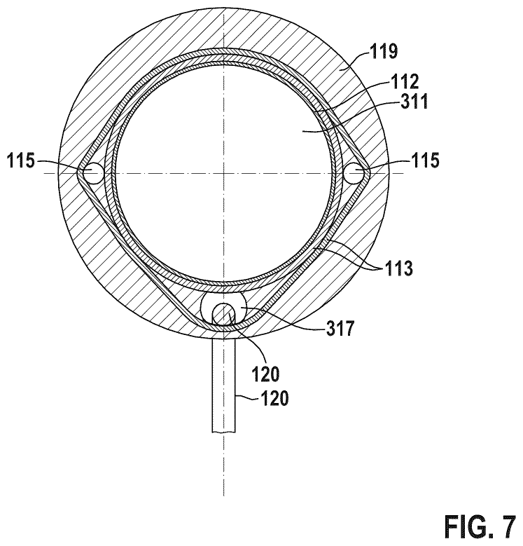

Then, the mandrel 111 and the solid wire 117 is removed from the assembly by pulling. The protruding end of the solid wire 117 may be clamped and pulled out of the shaft, preferably at a constant speed. The optimal speed is the highest speed at which the solid wire 117 does not yet break off but constricts and thus peels out of the shaft. Thereby an inner lumen 311 and a guide lumen 317 is created. Now, a steering wire 120 is introduced into the guide lumen 317 left by the solid wire 117 after its removal. FIG. 7 shows the construction of the assembly after this step.

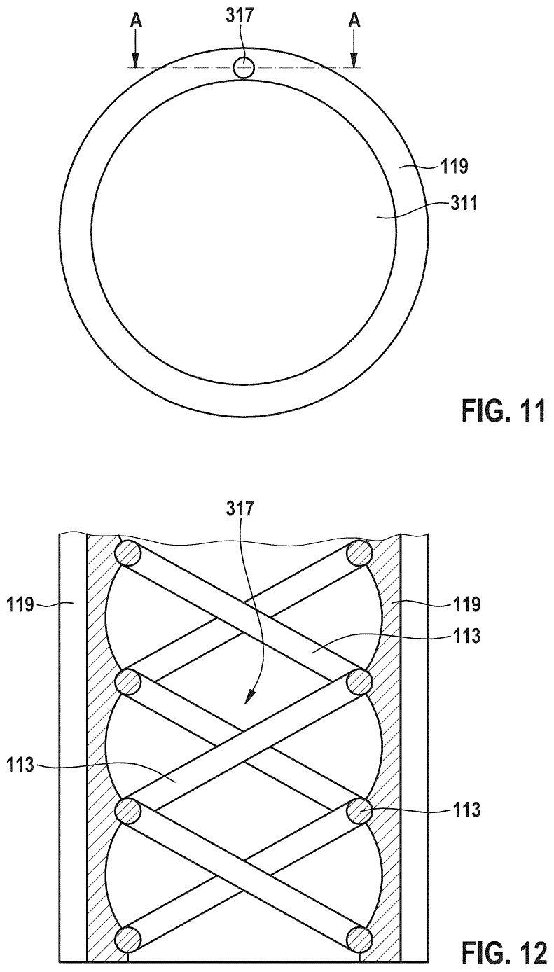

FIG. 11 shows the embodiment of the assembly of FIG. 6 after the manufacturing steps described above in a cross section, with the inner lumen 311 and the guide lumen 317. The steering wire 120 within in the guide lumen 317 is not shown here (but of course available in the final assembly).

FIG. 12 shows the assembly of FIG. 11 in a longitudinal section along A-A in FIG. 11 (size ratios in FIG. 12 and FIG. 11 differ, but do not change the basic principle). Thus, FIG. 12 shows also an inner surface of the guide lumen 317 that extends in longitudinal direction. As one can derive from this FIG. 12, the reinforcement elements 113 form helical or quasi-helical projections at the surface of the laminated material of the cover layer 119 after removal of the solid wire 117. This is because the laminated material of the cover layer 119 has adapted the surface structure of the reinforcement elements 113 and the solid wire 117, wherein the reinforcement elements 113 were pressed into the surface of the solid wire 117 during the previous manufacturing step thereby deforming the surface of the solid wire 117. Accordingly, a steering wire 120 running within the guide lumen 317 experiences only small friction forces thereby facilitating steering as the inner surface of the guide lumen 317 being in contact with the steering wire 120 is small. In FIG. 1 showing the catheter with the tubular device 1 the steering wire is referred to as reference number 9.

As indicated above, the grab 203 is configured such that the mandrel 111 may be rotated about its longitudinal axis (see arrow 203a in FIG. 15) also during the braiding step. Thereby the solid wire and the support wires extend at least partially around the mandrel and thereby the inner lumen when the mandrel is removed. For example, with regard to the first embodiment this is depicted in FIG. 14. This Fig. shows a cross section parallel to the one of FIG. 2 but at a different position with regard to the longitudinal direction. It is shown that the solid wire 17 as well as the support wires 15 extend across a circumferential angle a of, for example, 30° with reference to the cross section of FIG. 2. Thereby, a steerable catheter imparts sufficient dimensional stability (particularly torsional stability) to the tube so as to allow a relatively high lateral force to be exerted, into a distal end of the catheter tube, by a rotation of the actuator or the shaft. With regard to the catheter, thereby it is possible to exert comparatively high pressing forces onto the tissue.

The above method allows automatic and continuous manufacturing of assemblies for tubular devices for a steerable catheter or sheath. Additionally, such tubular device provides good steering properties since the friction forces acting at the steering wire are reduced.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A method of manufacturing a tubular device for a catheter sized for introduction into a patient's body, comprising the following steps:
Providing a mandrel having an outer sheath as well as a first end, a second end and a longitudinal axis;
Providing a solid wire consisting of elastic material;
Positioning an end section of the solid wire adjacent the first end of the mandrel such that it extends parallel to and/or around the longitudinal axis of the mandrel;
Wrapping one or more reinforcement elements around the mandrel and around at least a pre-defined section of the solid wire with such tension that the one or more reinforcement elements are pressed into the surface of the solid wire and simultaneously supplying further sections of the solid wire parallel to the longitudinal axis of the mandrel until the second end of the mandrel or a pre-defined section of the mandrel is covered by the one or more reinforcement elements;
Cutting through the solid wire and the one or more reinforcement elements and, if applicable, the mandrel, thereby receiving an assembly;
Providing a cover layer and laminating this cover layer and/or providing a cover layer material and extruding this cover layer material onto the assembly; and
Removing the solid wire by pulling and removing the mandrel pulling from the covered assembly.

2. The method of claim 1, wherein the outer sheath of the mandrel is a PTFE layer and/or comprises an anti-stick coating at its inner surface.

3. The method of claim 1, wherein the solid wire is an elastomer having a Young's modulus below 0,6 GPa, and a glass transition or melting temperature that is higher than the one of the cover layer material or the temperature used during the lamination and/or extrusion step.

4. The method of claim 1, wherein at least three reinforcement elements are used and provided by one or more rotating first horn gears of a braiding machine which may be configured such they rotate about their own central axis and about the longitudinal axis of the mandrel.

5. The method of claim 1, wherein the solid wire is provided by a second horn gear of a braiding machine, wherein the second horn gear is configured such that it is movable in radial direction with regard to the longitudinal axis of the mandrel.

6. The method of claim 1, wherein at least one support wire is provided simultaneously with the solid wire and parallel to and/or around the longitudinal axis of the mandrel.

7. The method of claim 1, wherein the assembly is inserted into at least one cover layer tube prior lamination.

8. The method of claim 1, wherein the assembly is inserted into several cover layer tubes or tube elements such that these tubes/elements are arranged adjacently in longitudinal direction, wherein at least two of these tubes have different materials or material properties.

9. The method of claim 1, wherein the solid wire or a guide lumen created by the solid wire extends helically around the longitudinal axis of the mandrel at or along a distal portion of the mandrel.

10. The method of claim 1, wherein the solid wire outside the reinforcement elements at or along a proximal portion of the mandrel.

11. An assembly for a tubular device for a catheter sized for insertion into a patient's body, comprising several layers, namely
a hollow cylindrical inner liner forming an inner lumen and having a longitudinal axis,
a braid or wrapped component of one or more reinforcement elements wrapped around the inner liner,
a cover layer surrounding the braid or wrapped component, and
a guide lumen,
wherein the guide lumen is provided parallel to and/or around the longitudinal axis, wherein the guide lumen is arranged between the inner liner and the cover layer and is wrapped by at least a pre-defined part of the one or more reinforcement elements, wherein the one or more reinforcement elements form projections at the inner surface of the guide lumen.

12. The assembly of claim 11, wherein the guide lumen extends in longitudinal direction along a pre-defined section of the inner lumen.

13. The assembly of claim 11, wherein the assembly comprises at least one support wire extending parallel to and/or around the longitudinal axis and is wrapped by at least a pre-defined part of the one or more reinforcement elements.

14. A tubular device for a steerable catheter sized for introduction into a patient's body comprising the assembly of claim 11, wherein a steering wire is located within the guide lumen.

15. The device of claim 14, wherein a distal end of the steering wire is fixed to the distal end of the catheter, wherein the catheter comprises an actuator at its proximal end, wherein the actuator is coupled to a proximal end of the steering wire such that actuation of the steering wire causes bending or change in direction of the distal end of the catheter.
